(19) United States
(12) Patent Application Publication
(10) Pub. No.: US 2009/0114847 A1
(43) Pub. Date: May 7, 2009

(54) PARTICLE THERAPY

(76) Inventors: Sven Oliver Grozinger, Herzogenaurach (DE); Klaus Hermann, Nurnberg (DE); Elke Rietzel, Darmstadt (DE); Andres Sommer, Langensendelbach (DE); Torsten Zeuner, Erlangen (DE)

Correspondence Address: BRINKS HOFER GILSON & LIONE, P.O. BOX 10395, CHICAGO, IL, 60610, US

(21) Appl. No.: 12/092,695

(22) PCT Filed: Nov. 9, 2006
(86) PCT No.: PCT/EP06/68308

§ 371 Date: May 15, 2008

(51) Int. Cl. A61N 5/00

(52) U.S. Cl. 250/492.1

(57) ABSTRACT

The invention relates to a treatment room for a particle therapy system that has a treatment room isocenter, which can be set variably during treatment and forms an origin of a coordinate system, and a patient positioning apparatus for automatically positioning the patient with reference to the set treatment room isocenter.

The present patent document is a nationalization of PCT/EP2006/068308, filed Nov. 9, 2006, designating the United States, which is hereby incorporated by reference. This application also claims the benefit of EP 05024743.6, filed Nov. 11, 2005, which is hereby incorporated by reference.

### BACKGROUND

The present embodiments relate to a particle therapy system for irradiating a volume of a patient that is to be irradiated with high energy particles. The present embodiments may also relate to the planning and carrying out of irradiation in a treatment room of a particle therapy system.

A particle therapy system has at least one treatment room with a beam exit from which a particle beam emerges in order to interact with the patient positioned in an irradiation position. Usually, the irradiation position is given with reference to an irradiation isocenter of the particle therapy system. Furthermore, the particle therapy system usually has an imaging apparatus for verifying the position of the target volume with reference to the particle beam, and a patient positioning apparatus with which, for the purpose of irradiation, the patient can be brought into the irradiation position.

Various irradiation systems and techniques are known from H. Blattmann in “Beam delivery systems for charged particles”, Radiat. Environ. Biophys. (1992) 31:219-231. A particle therapy system is disclosed, for example, in EP 0 986 070.

A particle therapy system usually has an accelerating unit and a high energy beam guiding system. Acceleration of the particles, for example, protons, pions, helium, carbon or oxygen ions, is performed, for example, with the a synchrotron or cyclotron.

The high energy beam transport system guides the particles from the accelerating unit to one or more treatment rooms. A distinction is made between fixed beam treatment rooms, in which the particles strike the treatment site from a fixed direction, and gantry-based treatment rooms. In the case of the latter, it is possible to direct the particle beam on to the patient from various directions.

A control and safety system of the particle therapy system ensures that a particle beam characterized by the requested parameters is guided into the appropriate treatment room. The parameters are defined in a treatment or therapy plan. The treatment or therapy plan specifies how many particles are to strike the patient from which direction and with which energy.

The therapy plan is usually generated with imaging methods. For example, a 3D data record is generated using a computed tomography unit. The tumor is localized inside this image data record, and the required radiation doses, directions of incidence, and types of particle are fixed.

During the irradiation, the patient is positioned in the irradiation position on which the therapy planning is based. This is performed, for example, using fixing masks. In addition, before the irradiation the patient's position is checked using an imaging device. In this case, the current irradiation position is matched to the image data record on which the therapy planning is based.

During this so called position verification, images from various directions are matched with, for example, projections from the CT planning data record before an irradiation. Fluoroscopic images are obtained for this purpose in the beam direction and orthogonal thereto. The recordings of these images are carried out in the irradiation position near the beam exit. Only limited space is available for imaging.

In general, there are imaging methods for obtaining 3D image data records which are based on the fact that fluoroscopies are carried out from various directions. 3D-type image data records can be obtained from the image data in a fashion similar to a CT picture. One possibility for such an imaging apparatus is an imaging robot that can be aligned freely about a patient to be X-rayed. X-raying the patient from various directions requires availability of appropriately sufficient space. Another possibility for obtaining 3D pictures is, for example, a C-arm X-ray machine.

Such imaging devices for obtaining 3D image data records require sufficient space to be able to X-ray the patient from various directions. It must be possible to move elements of the imaging unit about the patient in order to take images at an adequate spacing.

In general, the patient is positioned close enough to the beam exit to keep the expansion of the beam through scattering as slight as possible. A customary spacing between the isocenter of an irradiation site and the beam exit is approximately 60 cm.

The preferred spacing, addressed above, of the irradiation isocenter from the beam exit constrains the imaging of the position verification to imaging apparatuses that occupy correspondingly little space.

### SUMMARY AND DESCRIPTION

The present embodiments may obviate one or more of the drawbacks or limitations inherent in the related art. For example, in one embodiment, an irradiation of a patient is planned and carried out, such that the performance of a high precision therapy system that can be flexibly used, is exploited. In another example, in one embodiment, particle therapy has various types of particles by a scanning technique and with a highly accurate position verification. A particle therapy system t includes imaging techniques that take up space to be used in verifying position.

In one embodiment of the treatment room, the treatment room has a treatment room isocenter that can be set variably during treatment and forms an origin of a coordinate system, and a patient positioning apparatus for automatically positioning the patient with reference to the set treatment room isocenter. The treatment room isocenter for irradiation, such as the irradiation isocenter, can be set variably.

The treatment room isocenter is the origin of a coordinate system in the treatment room. Positionings, for example, of the patient support apparatus, of the patient, of an imaging unit and/or of a particle beam path are defined in the treatment room with reference to the isocenter. The isocenter's position along the particle beam path defines the beam parameters present in the irradiation, such as beam diameter and beam profile, in particular the steepness of the drop in the beam profile.

If the treatment room isocenter can be set, the treatment room isocenter is no longer fixed to a point in the treatment room, but that it can be selected and set freely, for example, in a fashion limited to one region. The treatment room isocenter can be identified in space by an appropriately alignable laser cross. In addition or as an alternative, the treatment room isocenter or its position or coordination in the treatment room may be stored as stored information in, for example, a therapy control center, and to make use of it in controlling an irradiation procedure. The stored information is transmitted, for example, to the positioning apparatus and/or used as a basis for driving the positioning apparatus when a therapy plan isocenter is to be set with reference to the treatment room isocenter. Furthermore, the stored information can be transmitted to an imaging unit and/or be used as a basis for driving the imaging unit when, for example, imaging is to be carried out in a fashion centered around the treatment room isocenter.

The beam parameters, such as beam diameter and beam profile, in particular, the steepness of the drop in the beam profile, are a function of the type of particle and the position of the treatment room isocenter in the beam path. The treatment room isocenter can be set in an appropriately flexible fashion.

An optimum spacing of the treatment room isocenter from a beam exit can be set in the treatment room for each irradiation procedure, for example, for each type of particle and for each irradiation direction. Together with an appropriately settable small beam diameter, such a beam then additionally has, for example, a steep radial drop in the particle distribution. A highly accurate and precise irradiation can be optimally repositioned. A raster scanning technique may be used to obtain the highly accurate and precise irradiation.

Furthermore, given an appropriate selection of the treatment room isocenter 3D imaging can also be carried out with an imaging apparatus that makes corresponding demands on space. A very precise irradiation with a particle beam can thereby be carried out with regard to the verification of position, since the verification of position is performed with 3D data records, or at least data records of 3D type.

In one embodiment of the treatment room, the settable treatment room isocenter can be set along a beam central axis of the particle beam, for example, for irradiation procedures. The beam central axis is, for example, the beam path given by the zero position of a raster scanning apparatus.

In one embodiment, the distance between the treatment room isocenter for irradiation and for imaging is equal to or less than 2 m and, if possible, less than 0.5 m, and so the verification of position can also be undertaken repeatedly when possible during an irradiation without great loss of time owing to long travel paths. Maintaining the distance is possible, for example, whenever the movement path of the patient is kept as small as possible, that is to say when, for example, the imaging device has, or virtually has the minimum spacing from the beam exit.

In one embodiment, a patient positioning system (apparatus) includes a robotically driven patient table. The patient positioning system (apparatus) is preferably driven by a therapy control unit of the particle therapy system. The parameters for carrying out a change in position can be stored in the therapy plan that forms the basis of the therapy control unit for controlling the irradiation.

In one embodiment, a therapy plan includes at least two procedures for irradiating and/or imaging with identical and/or different therapy plan isocenters, the procedures being assigned at least two spatially different treatment room isocenters. A therapy plan isocenter is a point (volume element) in an image data record of the patient to be irradiated which forms the basis of planning. With reference to this point, an irradiation procedure, for example, is planned. Geometric information for the irradiation, such as irradiation direction or a volume to be irradiated/imaged, is referred to this point. A relationship of the therapy plan isocenter to the treatment isocenter as is desired during the carrying out of the procedure is defined. Desired beam parameters that are given by the position of the treatment room isocenter are also taken into account during planning.

The relationship of the therapy plan isocenter to the treatment room isocenter, for example, laying the two isocenters one upon the other, is then produced during execution, for example, by positioning the patient or the imaging apparatus, and/or setting the treatment room isocenter appropriately.

A therapy plan is a data record that has been compiled, for example, with a computer unit and in which patient-related data are stored. By way of example, this can include a medical image of the tumor to be treated, and/or selected regions to be irradiated in the body of a patient, and/or risk organs whose radiation burden is to be kept as low as possible, and/or other information. Furthermore, by way of example this includes parameters that characterize the particle beam and that specify how many particles are to strike the patient or specific regions to be irradiated, from which direction and with which energy. The energy of the particles determines the depth of penetration of the particles into the patient, for example, the location of the volume element at which the maximum of the interaction with the tissue occurs during the particle therapy. In other words, the location at which the maximum of the dose is deposited. The therapy control unit can use the therapy plan to determine the control instructions required for controlling the irradiation. The therapy plan takes into account that the treatment room isocenter for the irradiation can be set variably.

Even when planning the therapy, the therapy freedom can be introduced into an optimized particle therapy via the spatial selection of the treatment room isocenters to be used, that is to say the isocenters spacing from the beam exit, for example. The verification of position can be performed independently, for example, of the position of a treatment room isocenter set for an irradiation, or treatment room isocenters can be selected as a function of the incidence angle and of the sorts of particles used.

### BRIEF DESCRIPTION OF THE DRAWINGS

FIG. 1 is a schematic of an embodiment of a particle therapy system,

FIG. 2 an exemplary flowchart for illustrating an irradiation procedure in accordance with a therapy plan, and

FIGS. 3 to 5 show schematics of a treatment room with variably settable treatment room isocenters.

### DETAILED DESCRIPTION

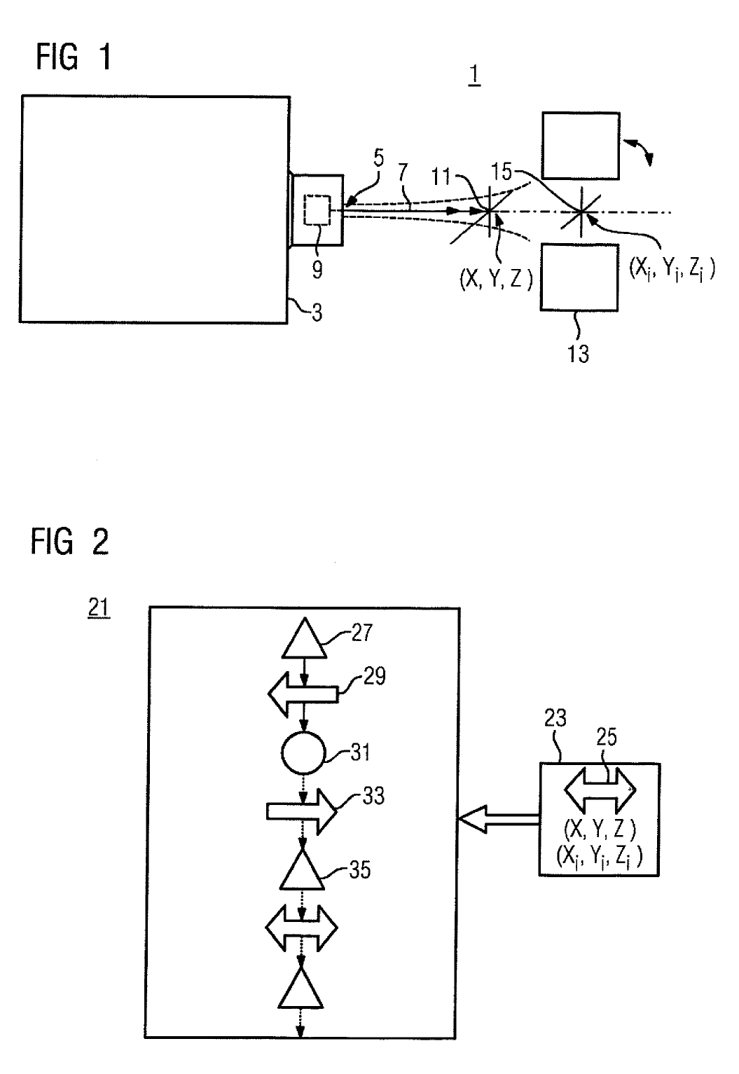

FIG. 1 shows a particle therapy system 1 for irradiating a volume, to be irradiated, of a patient with high energy particles. A particle accelerating unit 3 emits a particle beam 7 from a beam exit 5. The particle therapy system includes, for example, a raster scanning apparatus 9 that scans a scanning region of 20 cm×20 cm. A treatment room isocenter 11 may be set on a beam central axis that runs centrally in relation to the scanning region. The particle beam diverges because of scattering processes in the beam or with the matter being X-rayed. The closer a treatment room isocenter is arranged to the beam exit 5, the smaller the beam diameter of the particle distribution in the particle beam, and the more sharply defined the lateral drop in the particle distribution. A spacing of 60 cm may be selected in the case of irradiation with protons. At this spacing, the beam diverges to a desired beam diameter adopted in the therapy plan; for example, the irradiation is performed using a raster scanning method with a beam diameter of approximately 5 mm.

Furthermore, the particle therapy system 1 has an imaging apparatus 13 that may generate a 3D data record of the patient in the region of the volume to be irradiated. The imaging apparatus 13 is intended to be used to verify the position of the volume to be irradiated with reference to the particle beam. The imaging apparatus 13 has an imaging center 15. As a result of the design, such as the dimensions and structure, of the imaging apparatus 13, the spacing of the imaging center 15 from the beam exit 5 is greater than the spacing of the treatment room isocenter 11 provided for irradiation from the beam exit 5. For imaging purposes, a treatment room isocenter, such as the imaging center 15 in FIG. 1, is arranged on the beam central axis. The spacing between the treatment room isocenter 11 and the treatment room isocenter for imaging (imaging center 15) is kept as small as possible. For example, the spacing of the imaging center 15 from the beam exit 5 is 100 cm. A displacement of 40 cm in or against the beam direction can be carried out quickly and without stressing the patient even during an irradiation session.

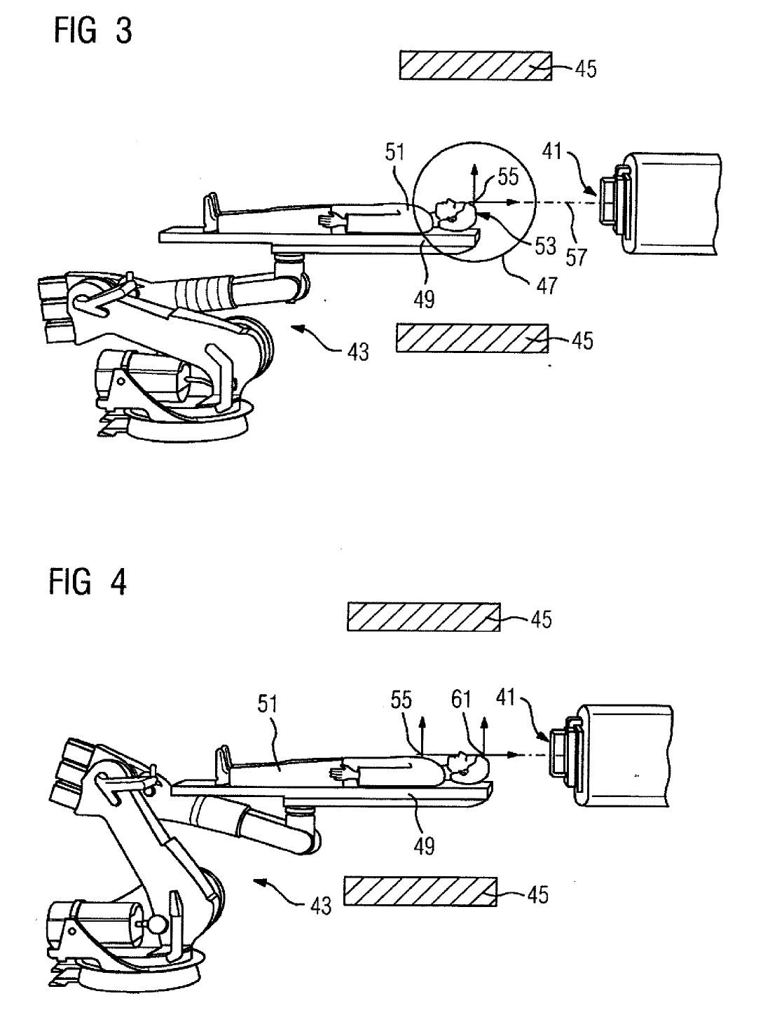

FIG. 2 shows an irradiation session 21 that is carried out on the basis of a therapy plan 23. In addition to the required beam parameters, the therapy plan 23 has the particle energy, the particle intensity, and direction of incidence, for various volume elements of the volume to be irradiated and for various irradiation procedures from various directions, for example. In addition, the therapy plan 23 includes information relating to the position (X, Y, Z) of the treatment room isocenters for irradiation, and/or the position (Xᵢ, Yᵢ, Zᵢ) of the treatment room isocenters for imaging, and/or possibly a displacement vector 25 that specifies by how much a patient or an imaging apparatus must be displaced so that therapy plan isocenters are matched with treatment room isocenters.

The irradiation session 21 may begin with a verification of position 27. Verification may positioning the patient in the imaging position in the treatment room isocenters for imaging (Xᵢ, Yᵢ, Zᵢ), in accordance with the therapy planning. Subsequently, a displacement 29 is carried out in accordance with the displacement vector 25. The patient is now in the irradiation position. A first irradiation procedure 31 is carried out in this position.

It however, the suspicion arises during the irradiation that the patient's position has changed, a second displacement 33 back into the imaging position can now be performed in order to carry out a further verification of position 35.

Such verifications of position can occur repeatedly because of suspected changes in position, for safety reasons, or in order to undertake a further irradiation, for example, from another direction of incidence.

The therapy plan 23 for the irradiation session 21, which possibly has a number of irradiation and/or imaging procedures, is performed, for example, in a number of acts. In one act, an imaging procedure is planned in which a therapy plan isocenter of the volume to be irradiated lies at the imaging center of the imaging apparatus. In this position (the imaging position), the imaging is to be carried out in order to verify the position of the patient in accordance with the irradiation planning. No beam is planned or applied in this imaging position.

An irradiation procedure is planned in another act. To this end, one or more treatment room isocenters are fixed, and one or more irradiation fields are planned. The planning of the irradiation procedure includes, for example, that at the beginning of the irradiation procedure the patient is positioned by the patient positioning apparatus such that the irradiation isocenter lies at an isocenter of the radiation location. An irradiation room isocenter is planned such that the patient is brought up as close as possible to the radiation exit without being in danger. For example, the treatment room isocenter is displaced from the imaging center to the position planned for the irradiation. The actual irradiation is then performed in this position (the irradiation position).

The treatment room isocenter for irradiation can be set variably.

Further imaging procedures and irradiation procedures, including under changed directions of incidence, depending on circumstances, may be planned. When use is made of a gantry, it is possible here for the different direction of beam incidence to require correspondingly matched treatment room isocenters.

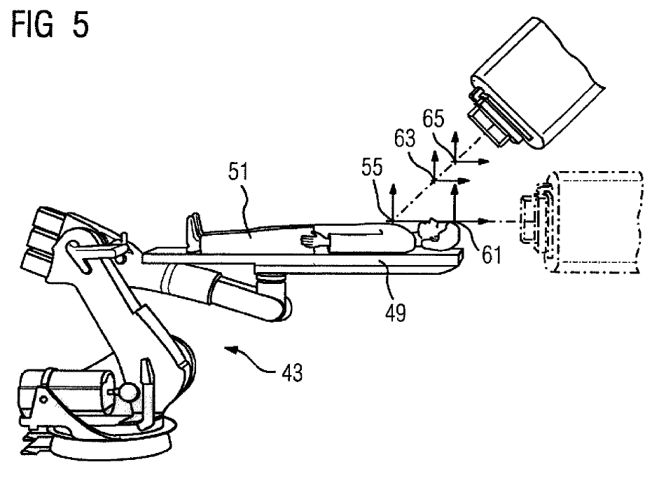

FIG. 3 shows an example of a treatment room with a beam exit 41, a patient positioning apparatus 43 and an imaging apparatus 45 with an imaging volume 47. The patient positioning apparatus 43 has a patient couch (support) 49 on which a patient 51 lies. The volume, to be irradiated, of the patient 51 lies, for example, inside a skull 53 of the patient 51. The imaging volume 47 has an imaging center 55. The imaging center 55 may be located on a beam central axis 57 of the particle beam, for example, at a distance of 100 cm from the beam exit 41. A picture, preferably a 3D picture (representation) of the volume to be irradiated, is recorded with the imaging device (apparatus) 45 for the purpose of verifying position. The treatment room isocenter is set to the position provided in the therapy plan. The settability of the treatment room isocenter enables the imaging apparatus to be planned in the positions required for 3D imaging.

The 3D picture is matched with pictures on which the therapy planning was based and, the patient 51 may be readjusted with the patient positioning apparatus 43 into the position on which the therapy planning is based. The patient is then located in the imaging position defined in the therapy plan.

The patient 51 is moved from the imaging position into the irradiation position that is illustrated in FIG. 4. The treatment room isocenter is set to the position envisaged in the therapy plan. The volume previously situated around the imaging center 55 and to be irradiated now lies around the irradiation isocenter 61 and can, for example, be irradiated with a (raster) scanning apparatus in a fashion specific to volume element.

In a departure from FIG. 4, in FIG. 5 the beam exit has been adopted as part of a gantry, and rotated by an angle into a further irradiation position with another angle of incidence. A similar situation can be obtained for a treatment center with two beam exit possibilities. A treatment room isocenter 63 is indicated for irradiation with, for example, protons from this angle, and a treatment room isocenter 65 is indicated for irradiation with carbon ions. The treatment room isocenters can be optimized to the types of particles at the spacing from the beam exit. If the therapy plan includes an irradiation procedure with one of these types of particles at this angle, the patient is moved for irradiation such that the associated therapy plan isocenter is matched with the treatment room isocenter. The imaging unit is moved to this end, and the positioning unit 43 is driven in accordance with the respective treatment room isocenter.

1. A treatment room in a particle therapy system, the treatment room comprising:
a treatment room isocenter for irradiation, which can be set variably during treatments and forms an origin of a coordinate system, and
a patient positioning device for automatically positioning the patient with reference to the set treatment room isocenter.

2. The treatment room as claimed in claim 1, further comprising a beam exit of a beam guiding and accelerating system from which a particle beam emerges to interact with a patient positioned in an irradiation position, the irradiation position being given by the position of the set treatment room isocenter for irradiation.

3. The treatment room as claimed in claim 1, characterized in that a distance of the treatment room isocenter for irradiation can be set relative to the beam exit as a function of the particle, such as protons, carbon ions or oxygen ions.

4. The treatment room as claimed in claim 1, characterized in that the variable treatment room isocenter can be set variably on a beam central axis of a particle beam running in the treatment room.

5. The treatment room as claimed in claim 1, characterized in that at least one beam parameter such as a beam width, a beam profile, a falling edge of the beam profile, or a combination thereof, can be set for an irradiation procedure by selecting the position of the variable treatment room isocenter on the beam central axis.

6. The treatment room as claimed in claim 1, characterized in that at least one parameter characterizing the particle beam, such as beam focus, beam divergence, beam diameter in the treatment room can be set.

7. The treatment room as claimed in claim 1, characterized in that the patient positioning apparatus comprises a robotically driven patient table that can be driven by a therapy control unit of the particle therapy system in order to move the patient from an imaging position to an irradiation position.

8. The treatment room as claimed in one claim 1, characterized in that a controllable laser marks the set position of the variable treatment room isocenter in the treatment room.

9. The treatment room as claimed in claim 1, further comprising: an imaging device for verifying the position of the volume to be irradiated with reference to the particle beams is the imaging device is operable to verify the position of the volume to be irradiated in an imaging position of the patient, the imaging position being given by the position of the set treatment room isocenter and being arranged at a distance from the irradiation position in space.

10. The treatment room as claimed in claim 9, characterized in that the imaging position can be assigned an imaging center that is arranged on the beam central axis.

11. The treatment room as claimed claim 9, characterized in that the imaging apparatus is operable for 3D imaging.

12. The treatment room as claimed in claim 9, characterized in that a space that is available for the imaging unit can be set by setting the variable treatment room isocenter on the beam central axis.

13. The treatment room as claimed claim 9, characterized in that the imaging device has dimensions that define a minimum spacing from the beam exit, and that the imaging device is arranged at least at this minimum spacing from the beam exit, the minimum spacing being greater than the distance between the beam exit and irradiation isocenter.

14. The treatment room as claimed in claim 9, characterized in that the imaging device is a C arc X-ray machine or an imaging robot that, for the purpose of 3D imaging, is designed to rotate about the imaging position, about the imaging center, and that a minimum spacing from the beam exit is determined by the rotatability, and the imaging device is arranged at least at this minimum spacing from the beam exit.

15. The treatment room as claimed in claim 1, further comprising a therapy plan for irradiating the patient, the therapy plan including
at least two procedures for irradiating and/or imaging with identical and/or different therapy plan isocenters, the at least two procedures being assigned to at least two spatially different treatment room isocenters.

16. A method for drawing up a therapy plan, the method comprising:
determining a radiation dose distribution for an irradiation procedure using a database in which characteristic beam parameters for various treatment room isocenters are stored as a function of the spacing of the treatment room isocenter from a beam exit or beam focus.

17. The method as claimed in claim 16, wherein treatment room isocenters are assigned in a therapy plan to irradiation procedures from different irradiation directions and/or with different types of particle.

18. The method as claimed in claim 16, wherein the irradiation procedure is planned for a particle irradiation that uses a scanning technique, such as a raster scanning technique.

19. An irradiation method for irradiating a volume of a patient that is to be irradiated with high energy particles of a therapy system having at least two procedures for irradiating and/or imaging identical or different therapy plan isocenters in a treatment room, wherein the therapy plan isocenters of at least two of the procedures are assigned spatially different treatment room isocenters, the treatment room isocenter is reset between procedures that are assigned spatially different treatment room isocenters, and the patient is respectively positioned for carrying out the procedures in such a way that the respectively planned therapy plan isocenters and the respectively set treatment room isocenter are matched.

20. The irradiation method as claimed in claim 19, wherein various treatment room isocenters are assigned to irradiation procedures from different irradiation directions and/or with different types of particle.

21. The irradiation method as claimed in claim 19, wherein at least one position changing operation the patient's position is changed between two spatially different treatment room isocenters by driving a patient positioning unit, in particular in which the patient is moved in or against the irradiation direction in order to change the position of the position changing operation.

22. A particle therapy system having a treatment room, the treatment room including:
a treatment room isocenter for irradiation, which can be set variably during treatment, and forms an origin of a coordinate system, and
a patient positioning device for automatically positioning the patient with reference to the set treatment room isocenter.
